(19) United States
(12) Patent Application Publication
(10) Pub. No.: US 2013/0052708 A1
(43) Pub. Date: Feb. 28, 2013

(54) METHOD FOR CONVERSION OF CARBOHYDRATE POLYMERS TO CHEMICAL PRODUCTS USING CERIUM OXIDE CATALYST

(71) Applicants: Chin Li Cheung, Lincoln, NE (US); Neil J. Lawrence, Lincoln, NE (US); Allen Kruse, Beatrice, NE (US)

(72) Inventors: Chin Li Cheung, Lincoln, NE (US); Neil J. Lawrence, Lincoln, NE (US); Allen Kruse, Beatrice, NE (US)

(21) Appl. No.: 13/565,359

(22) Filed: Aug. 2, 2012

Related U.S. Application Data
(60) Provisional application No. 61/515,054, filed on Aug. 4, 2011.

(51) Int. Cl. C13K 1/00; C12P 7/06; B01J 27/053; B01J 27/055; B82Y 40/00

(52) U.S. Cl. 435/161; 502/217; 502/218; 127/37; 977/896

(57) ABSTRACT

Methods are described for conversion of carbohydrate polymers, including cellulose, that yield monosaccharide products, including glucose. Catalyst compositions that include functionalized metal/metal oxide clusters on cerium oxide nanostructures are described which provides product yields, e.g., greater that 50% in a single step process.

### CROSS-REFERENCE TO RELATED APPLICATIONS

This application claims priority to provisional application Ser. No. 61/515,054, which was filed on Aug. 4, 2011, and which is incorporated herein by reference in its entirety.

### TECHNICAL FIELD

The invention generally relates to conversion of carbohydrates, and more particularly to the conversion of carbohydrate polymers (such as cellulose or cellobiose) using cerium oxide catalysts to obtain valuable chemical products such as glucose.

### BACKGROUND

Cellulose is a complex polymer consisting of a linear chain of several hundred to over ten thousand β1,4 linked D-glucose units. Over 1.3 billion tons of cellulose is produced in the United States each year, making it an abundant biomass resource capable of displacing 30% of the annual petroleum consumption in the United States. To convert cellulose to other fuels, hydrolysis is necessary to obtain monomer building blocks from which desired chemicals (such as ethanol) may be derived. The hydrolysis reaction is strongly affected by structural and compositional features such as crystallinity and polymer chain length, all of which affect the desired product yields. At present, depolymerization is a recognized bottleneck in the conversion of cellulose feeds. While considerable research effort has been aimed at improving cellulose depolymerization processes in aqueous systems, progress has been limited, in part, due to the lack of solubility of cellulose in water. Enzymatic hydrolysis of cellulose is effective but is characteristically slow at ambient temperatures, and is also sensitive to contaminants originating from the various biomass components. Mineral acids have been extensively investigated to catalyze hydrolysis at a variety of acid concentrations and temperatures, but degradation of resulting products continues to be an issue. Accordingly, new methods are needed for converting carbohydrate polymers, such a cellulose and cellobiose, to value-added chemicals.

### SUMMARY

Applicants have discovered that acid catalysts containing sulfated metal oxide nanoparticles on cerium oxide nanorods with engineered oxygen defects can yield effective conversion of cellulose and cellobiose to glucose with greater than 50% yield and greater than 90% selectivity. Accordingly, the present invention is a process for conversion of carbohydrate polymers to useful end products such as glucose. The process includes: heating the carbohydrate polymer with a cerium oxide catalyst for a time sufficient to convert the carbohydrate polymer to the monosaccharide product.

Carbohydrate polymers include cellulosic polymers such as cellulose, hemicellulose, or cellobiose or other carbohydrates such as dextran, fructan or agarose. The monosaccharide reaction product includes, e.g., glucose, fructose, or galactose. Reaction processes described herein employ aqueous, organic or ionic liquids in the reaction medium and various cerium oxide structures as reaction catalysts. The concentration of carbohydrate polymers in the reaction medium is preferably in the range of about 30 to about 60 wt % in solution. Temperatures for conversion are preferably in the range of 50 to 200° C. Time to achieve conversion of the carbohydrate polymers is preferably a time in the range of about 0.01 hours to about 8 hours.

The cerium oxide catalyst may comprise a fluorite lattice structure containing cerium atoms in mixed valence states of Ce³⁺ and Ce⁴⁺, in which the ratio of Ce³⁺/(Ce³⁻+Ce⁴⁺) in the lattice ranges from 40% to 90% at 20° C., the valence states Ce³⁺ and Ce⁴⁺ are reversible in reduction and oxidation reactions, and the cerium oxide catalyst comprises small particles decorated near a surface of the fluorite structured cerium oxide lattice, in which the surface region of the cerium oxide lattice structure has a higher concentration of the small particles than an inner region of the cerium oxide lattice structure, the small particles having a diameter equal to or less than 1 nm. In various embodiments, the cerium oxide is sulfated and/or decorated with small particles comprising at least one of gold, silver, tin, palladium, platinum, an alloy of gold and silver, an alloy of gold and copper, the oxide of any the above, or a combination of any of the above. In some embodiments, the concentration of the small particles on the fluorite structured cerium oxide ranges from 0.001 to 5.0 atomic percent compared to cerium. In some embodiments, the small particles comprise tin particles, and the concentration of the tin particles on the cerium oxide ranges from 0.001 to 5.0 atomic percent compared to cerium. The concentration of the tin particles may also range from 0.005 to 0.02 atomic percent compared to cerium. In some embodiments, the small particles comprise at least one of tin particles or tin oxide particles, and the concentration of the tin particles or tin oxide particles on the cerium oxide ranges from 0.1 to 5 atomic percent compared to cerium.

In some embodiments, the cerium oxide comprises cerium oxide nanoscale structures, including nanotubes, nanocubes, nanoparticles, nanorods, nanowires, nanostars or complex nanoshapes. In some another embodiment, the ratio of Ce^{3|}/(Ce^{3|}+Ce^{4|}) in the lattice structure ranges from 40% to 50% at 20° C., or from 50% to 60%, or from 60% to 70%, or from 70% to 90% at 20° C. In other embodiments, the cerium oxide is capable of maintaining effective catalytic ability at temperatures at least up to 450° C.

The invention also provides methods of fabricating the cerium oxide catalyst comprising: fabricating fluorite structured cerium oxide having a lattice structure comprising cerium atoms in mixed valence states of Ce³⁺ and Ce⁴⁺; decorating the cerium oxide with small particles near a surface of the lattice structure such that a surface region of the cerium oxide lattice structure has a higher concentration of the small particles than an inner region of the cerium oxide lattice structure, the small particles having a diameter less than 1 nm; sulfating the cerium oxide; and activating the cerium oxide, in which after activation, the ratio of Ce³⁺/(Ce³⁺+Ce⁴⁺) in the cerium oxide lattice structure ranges from 40% to 90% at 20° C., the valence states Ce³⁺ and Ce⁴⁺ being switchable in reduction and oxidation reactions.

A more complete appreciation of the invention will be readily obtained by reference to the following description and the accompanying drawings.

### DESCRIPTION OF DRAWINGS

FIG. 1 is a diagram of a process for producing cerium oxide nanotubes by hydrothermal synthesis and Kirkendall diffusion.

FIGS. 2A and 2B are transmission electron microscopy images of a cerium oxide nanotube.

FIG. 3 is a graph showing the carbon monoxide conversion efficiency of different forms of cerium oxide at various temperatures.

FIG. 4 is an HPLC-MS chromatograph of a cellobiose sample prior to addition of a 0.01% Sn/SO₄/CeO₂ catalyst.

FIG. 5 is an HPLC-MS chromatograph of a cellobiose sample after 4 hours of digestion with a 0.01%Sn/SO₄/CeO₂ catalyst.

FIG. 6 shows Extended x-ray absorption fine structure spectroscopy (EXAFS) data for bulk CeO₂ and CeO₂ nanoparticles and nanorods.

FIG. 7 depicts Ramen scattering data of bulk CeO₂ and CeO₂ nanoparticles and nanorods.

### DETAILED DESCRIPTION

Described here is a process and catalyst composition for conversion of cellulose and other carbohydrate polymers to useful monosaccharides such as glucose. The process includes: heating the carbohydrate polymer with a cerium oxide catalyst for a time sufficient to convert the carbohydrate polymer to the monosaccharide product, the cerium oxide catalyst having a fluorite lattice structure comprising cerium atoms in mixed valence states of Ce^{3|} and Ce^{4|}, in which the ratio of Ce^{3|}/(Ce^{3|}+Ce^{4|}) in the lattice ranges from 40% to 90% at 20° C., the valence states Ce³⁺ and Ce⁴⁺ being reversible in reduction and oxidation reactions, the cerium oxide catalyst comprising small particles decorated near a surface of the fluorite structured cerium oxide lattice, in which the surface region of the cerium oxide lattice structure has a higher concentration of the small particles than an inner region of the cerium oxide lattice structure, the small particles having a diameter equal to or less than 1 nm.

The carbohydrate polymers include natural polysaccharides, such as starches and cellulosics. Exemplary carbohydrate polymers include, but are not limited to, e.g., cellulose, hemicellulose, cellobiose, maltodextrin, starch, or other selected carbohydrates such as, agarose, hyaluronic acid, chitin, acyl gellan, dextran, carboxymethylcellulose, carboxymethyl starch, carboxymethyl chitin, poly(lactide-co-ethylene glycol).

The monosaccharide reaction products include radicals of cladinose, allose, altrose, arabinose, erythrose, erythrulose, fructose, D-fucitol, L-fucitol, fucosamine, fucose, galactosamine, D-galactosaminitol, galactose, glucosamine, glucosaminitol, glucose, glyceraldehyde, glycerol, glycerone, gulose, idose, lyxose, mannosamine, annose, psicose, quinovose, quinovosamine, rhamnitol, rhamnosamine, rhamnose, ribose, ribulose, sorbose, tagatose, tartaric acid, threose, xylose and xylulose. The monosaccharide may further be a deoxy sugar (alcoholic hydroxy group replaced by hydrogen), amino sugar (alcoholic hydroxy group replaced by amino group), a thio sugar (alcoholic hydroxy group replaced by thiol, or C═O replaced by C═S, or a ring oxygen of cyclic form replaced by sulfur), a seleno sugar, a telluro sugar, an aza sugar (ring carbon replaced by nitrogen), an imino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group), aldonic acid (aldehydic group replaced by carboxy group), a ketoaldonic acid, a uronic acid, an aldaric acid, and so forth Amino sugars include amino monosaccharides, preferably galactosamine, glucosamine, mannosamine, fucosamine, quinovosamine, neuraminic acid, muramic acid, lactosediamine, acosamine, bacillosamine, daunosamine, desosamine, forosamine, garosamine, kanosamine, kansosamine, mycaminose, mycosamine, perosamine, pneumosamine, purpurosamine, rhodosamine. It is understood that the monosaccharide and the like can be further substituted. In some embodiments the monosaccharide is further processed in order to obtain other useful chemical products. For example, the monosaccharide reaction product glucose may be processed by fermentation to obtain ethanol.

Reaction processes described herein can employ solvents such as aqueous solvents, organic solvents, or ionic liquids, or mixtures thereof, as the reaction medium. Aqueous solvents include solvents containing water or solvents in which polar or hydrophilic compounds are preferably and substantially soluble. Organic solvents include protic or aprotic solvents, such as alcohols, esters, alkanes, ethers and aromatics. Suitable organic solvents include, e.g., benzene, tetrahydrofuran (THF), DMSO, methanol, ethanol, isopropyl alcohol and acetonitrile. Ionic liquids are salts that have a melting point, or that are liquid at, temperatures below about 200° C. Ionic liquids used in conjunction with the present invention comprise, e.g., 1-R₁-3-R₂-imidazolium compounds, where R₁ and R₂ are alkyl groups of formula (CₓH₂ₓ₊₁) where X=1 to 18. Exemplary ionic liquids include, but are not limited to, e.g., 1-ethyl-3-methylimidazolium chloride ([EMIM]Cl); 1-butyl-3-methylimidazolium chloride ([BMIM]Cl), 1-ethyl-3-methylimidazolium bromide ([EMIM]Br), 1-ethyl-3-methylimidazolium acetate ([EMIM]OAc), and combinations thereof. Nomenclature used herein to denote ionic liquids identifies the cationic portion of the ionic liquid, e.g., 1-ethyl-3-methyl-imidazolium, by bracket, e.g., [EMIM] or [EMIM]⁺. The anionic portion of the ionic liquid, e.g., halides (e.g., Cl or Br; or Cl⁻ or Br⁻) or acetates (e.g., ⁻OAc) is identified by placement outside the bracket (e.g., [EMIM]Cl or [EMIM]⁺Cl⁻). Unless otherwise noted, nomenclature for ionic liquids with or without ionic charges is used interchangeably, e.g., [EMIM]⁺Cl⁻ or [EMIM]Cl. In some embodiments of the invention, the ionic solvent is [EMIM]acetate.

Temperatures and reaction times are selected to maximize the selected carbohydrate products and to minimize product degradation. Temperatures for conversion are preferably in the range from about 50° C. to about 200° C. In some embodiments, the temperature for conversion is about 80° C. Time to achieve conversion of carbohydrate polymers is preferably a time in the range from about 0.01 hours to about 8 hours, but is not limited. The reactions may be carried out under acidic conditions at pH 1-5, but can also occur at higher pH values (pH 4-9). The reactions may proceed at atmospheric pressures, mild pressure or high pressure (up to 30 atm) conditions.

The reaction process described herein employs heating the carbohydrate polymer at a concentration of about 1-60 wt % in solution. In one embodiment, the carbohydrate is reacted at a concentration of about 30 wt % in solution. The monosaccharide products are obtained with high yields, e.g., up to about 50%, 60%, 70%, 80%, or 90%, or preferably about 100%, preferably in a single process step. The conversion of cellulose and other complex carbohydrates also exhibits high selectivity for desired chemical products (such as glucose), achieving up to about 60%, 70%, 80%, or 90% or about 100% selectivity.

The catalysts described herein include catalytic systems of cerium oxide (CeO₂) (also referred to as ceria). Cerium is a rare earth metal of the lanthanide series. While most of the rare earth metals exist in a trivalent state (+3), cerium exists in both a trivalent state and a tetravalent (+4) state and may switch between the two in reduction and oxidation reactions. Cerium(IV) oxide can be reduced by but not limited to carbon monoxide or other reducing agent such as H₂ to cerium(III) oxide:

2 CeO₂+CO→Ce₂O₃+CO₂,

or

2 CeO₂+H₂→Ce₂O₃+H₂O,

and cerium(III) oxide can be oxidized to cerium(IV) oxide by oxygen or other oxidizing agents:

2 Ce₂O₃+O₂→4 CeO₂.

As a result of alterations in cerium oxidation state, cerium oxide forms oxygen vacancies or defects in the crystal lattice structure by loss of oxygen and/or its electrons. The valence and defect structure of cerium oxide is dynamic and may change spontaneously or in response to physical parameters such as temperature, presence of other ions, and partial pressure of oxygen. These properties, combined with the abundance of cerium on earth, make ceria a low-cost and effective alternative to noble metal catalysts.

The presence of intra- and inter-molecular hydrogen bonding between the carbohydrate polymer chains (as in polymers such as cellulose) leads to the formation of highly complex geometric structures. Such structures pose geometric constraints for the catalyst, making it difficult for the catalyst to be positioned within sufficient spatial proximity to catalyze the breakage of glycosidic bonds. Conventional catalyst systems, for example, are only able to access terminal or superficial sections of the structures.

Applicants surprising discovered that the catalysts described here show excellent catalytic activity for the hydrolysis of carbohydrate polymers, such as cellulose and cellobiose. Without being bound by any theory of the invention, it is believed that the catalysts show exceptional activity for carbohydrate polymers due to their ability to detangle carbohydrate polymer chains and break the hydrogen bonding interactions. This allows the polymer to be converted to monosaccharaides with high efficiency at a large scale. The cerium oxide catalysts exhibit low aggregation susceptibility and provide better contact with fibrous cellulose substrates.

Cerium oxide having a fluorite lattice structure having cerium atoms in mixed valence states of Ce³⁺ and Ce⁴⁻, in which the ratio of Ce³⁺/(Ce³⁺+Ce⁴⁺) (referred to as the Ce³⁺ fraction) in the lattice ranges from about 0.40 to 0.70 or 40% to 70% can be fabricated by intentionally introducing defects into the ceria lattice. In some embodiments, the cerium oxide is activated in a low pressure oxygen containing environment to increase the defect sites in the ceria lattice. For example, the activation can be performed at a pressure of, e.g., 1 to 100 milli-Torr. In some embodiments, the cerium oxide is decorated with nanoparticles, such as tin nanoparticles, to enhance the catalytic ability of the cerium oxide. In some examples, the concentration of tin can range from about 0.001 to 5.0 atomic percent compared to cerium.

In the description below, catalysis will be defined as an increase in the rate at which equilibrium of a reaction is achieved through the addition of a substance (i.e., the catalyst) that, once the reaction is at equilibrium, is indistinguishable from its original form.

The catalytic activity of cerium oxide may be attributed to the effect of defects in the ceria lattice structure. One type of lattice defects that may affect the reactivity of the cerium oxide surface is oxygen vacancy defects, in which an oxygen atom is missing in the lattice. The degree of oxygen mobility in the ceria lattice can be attributed to the size, dispersion, and quantity of the oxygen vacancy defects. It appears that the fraction of cerium in the 3⁺ oxidation state can be used as a parameter to compare the relative densities of oxygen vacancy defects in different ceria samples.

In some embodiments, cerium oxide can be made in bulk form (which may have particles having dimensions in the micro meter scale), or as nanoparticles (having dimensions in the nanometer scale) or nanotubes (having tube structures with diameters in the nanometer scale). Fluorite structured cerium oxide nanotubes and nanorods may have larger surface areas, compared to cerium oxide nanoparticles or cerium oxide in bulk form. The Fluorite structured cerium oxide nanotubes and nanorods can be engineered to have a wide variety of defects with an emphasis on oxygen vacancy defects.

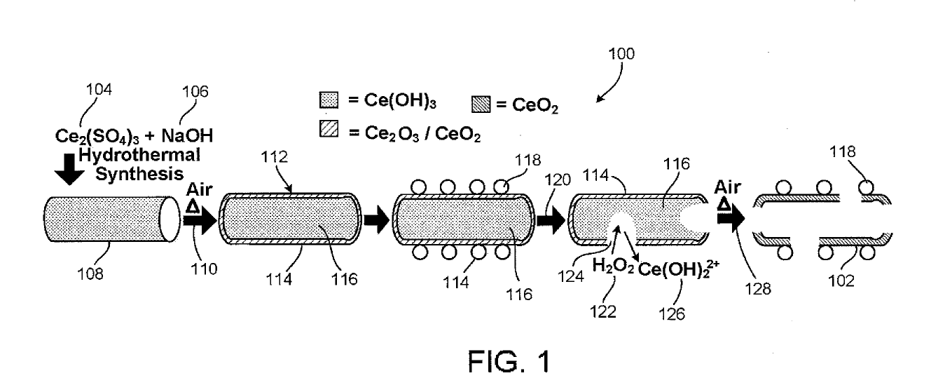

Referring to FIG. 1, in some embodiments, fluorite structured metal/metal oxide cerium oxide nanotubes 102 can be generated using a process 100 in which hydrothermal synthesis is used to generate nanowires, and Kirkendall diffusion mechanism is used to convert the nanowires to nanotubes. In Kirkendall diffusion, unequal diffusion rates in solids results in core-shell nanostructures and hollow nanostructures.

In the process 100, cerium(III) sulphate hydrate (Ce₂(SO₄)₃.X H₂O) 104 is dissolved in a sodium hydroxide solution (NaOH (aq)) 106 to form cerium (III) hydroxide (Ce(OH)3) nanorods and nanowires 108 under hydrothermal conditions. The cerium (III) hydroxide (Ce(OH)₃) nanorods and nanowires 108 are subjected to rapid oxidation 110 at 50° C. in a convection oven. The rapid oxidation leads to the formation of a core-shell material 112 with cerium oxide (CeO₂₋ₓ) forming an outer shell 114 of the cubic fluorite structure and a core 116 made of remaining Ce(OH)₃ in a simple hexagonal structure. The rapid conversion between the two crystalline structures produces many defects on the surface of the core-shell material 112.

The core-shell material 112 is mixed with an aqueous solution containing metal in an oxidized state 120 (e.g., SnCl₂), to facilitate an auto-reduction reaction. The core-shell material 112 has many Ce³⁺ ions that are oxidizable and react with metal in an oxidized state 120 in which the metal is reduced, resulting in metal nanoparticles 118 being deposited on the surface of the core-shell material 112.

This core-shell material 112 is sonicated in hydrogen peroxide (H₂O₂) 122, causing cracks 124 to be formed in the core-shell material and faster diffusion of the core material Ce(OH)₃ 126 away from the core 116 compared to diffusion of shell material CeO₂₋ₓ away from the shell 114. The samples are heated in a convection oven 128, resulting in the formation of fluorite structured cerium oxide nanotubes that are decorated with metal nanoparticles 102.

The metal decorated cerium oxide nanotubes are activated by oxidizing the nanotubes with air, then reducing the nanotubes with hydrogen. This modifies (e.g., increases) the ratio of Ce³⁺ to Ce⁴⁺ and reduces the metal.

The following describes an example in which fluorite structured tin cerium oxide nanotubes 102 were produced. Note that various parameter values, such as the quantities of the materials, temperatures of the processes, and time durations of the processes, are provided as examples only, other values can also be used.

A sample of 0.5 g cerium(III) sulphate hydrate (Ce₂(SO₄)₃.X H₂O, available from Sigma-Aldrich, St. Louis, Mo.) was dissolved into 40 mL of 10 M sodium hydroxide solution (NaOH (aq)). The solution was transferred to a 45 mL total volume Parr autoclave for hydrothermal treatment, and was allowed to react at 120° C. for 15 hours to produce cerium (III) hydroxide (Ce(OH)₃) nanorods and nanowires. The product was cooled and filtered using 3.0 μm membranes (available from Millipore, Billerica, Mass.) and rinsed with 3 aliquots of 50 mL water. All water used in this example was Ultrapure water of >18 MΩ resistivity filtered through 0.22 micron pore-sized filters.

After rinsing, the Ce(OH)₃ nanorods and nanowires were placed in a convection oven at 50° C. for 1 hour for partial oxidation to transform the P63/m Ce(OH)₃ into a cerium oxide containing both P 3̅m1 Ce₂O₃ and Fm 3̅m (fluorite) CeO₂₋ₓ. The partially oxidized sample was gently powdered using a spatula and heated in a convection oven at 50° C. for an additional hour for continued partial oxidation. The sample was treated with 50 mL water and an appropriate volume of an aqueous solution of tin chloride (10 ppm) for 5 minutes at room temperature after which 50 mL of 30% H₂O₂ the solution was sonnicated for 30 minutes followed by a further reaction time while stirring for 60 minutes. The resulting product was filtered using 0.8 μm Millipore membranes, rinsed with three 50-mL aliquots of water, and dried in a convection oven at 50° C. for 2 hours, resulting in the formation of fluorite structured tin cerium oxide nanotubes.

A 100 mg sample of the tin cerium oxide nanotubes was activated by thermal heating in a 1-inch quartz tube furnace with a 100 standard cubic centimeter per minute (sccm) flow of a nitrogen-oxygen mixture (80% N₂ and 20% O₂) for 1 hour at 350° C. under vacuum with an operating pressure of 0.1 Torr. The cerium oxide nanotubes produced using the process described above can have a diameter of about 20 nm and a length ranging from a few tens of nanometers to more than a micron.

As will be appreciated by those of ordinary skill in the art, the foregoing examples may be adapted to prepare cerium oxide nanotubes that are decorated with other metallic nanoparticles. For example, the above synthesis can be adapted to prepare cerium oxide nanotubes decorated with gold, silver, palladium, platinum, or nickel, by treating with auric acid, silver chloride, KPdCl₄, KPtCl₆, or nickel chloride, respectively.

In the examples above, the cerium oxide was decorated with metal particles. This is different from doping the cerium oxide with metal particles. There is a distinction between dopants and decorations. A dopant is an atom that is within the lattice of the host material, while a decoration is an atom, a molecule, or a cluster of atoms that may have a separate and distinct lattice and is found at or near the surface of the primary structure.

In order to convert the decorated cerium oxide nanoparticles into a solid superacid, the surface may be sulfated by treatment with sulfuric acid. For example, ceria (1 g) may be added to a diluted sulfuric acid (0.5 M) with stirring for 30 min, followed by filtration of the ceria and subsequent washing and drying of the ceria. In some embodiments, the decorated cerium oxide nanoparticles are modified iwht phosphoric acid functional groups.

Pre and post H₂O₂ treated ceria prepared herein was examined by X-ray diffraction (XRD) analysis using Bruker AXS D8 Discover with GADDS area, available from Bruker AXS Inc., Madison, Wis., for examining the crystallinity and crystal structure of the samples produced in different stages of the synthesis (data not shown). The weighted average wavelength of the Cu Kα x-ray source used was 1.5417 Å. The XRD spectrum was indexed to JCPDS 00-34-0394 Fm 3̅m CeO₂ and compared with JCPDS 00-023-1048 P 3̅m1 Ce₂O₂ and JCPDS 01-074-0665 Ce(OH)₃ P63/m.

The XRD analysis indicates that the ceria progressed from Ce(OH)₃ prior to oxidation with the H₂O₂ to a combination of Ce₂O₃ and CeO₂ (both fluorite structure) after treatment with H₂O₂, and finally to a match to just fluorite structured CeO₂ after the final calcination and activation step. The analysis showed the low pressure activated ceria nanotubes have pure Fm 3̅m cubic structure.

The process described above for fabricating metal-decorated cerium oxide nanotubes can be easily scaled to produce large batches of cerium oxide nanotubes that are robust over long periods of time.

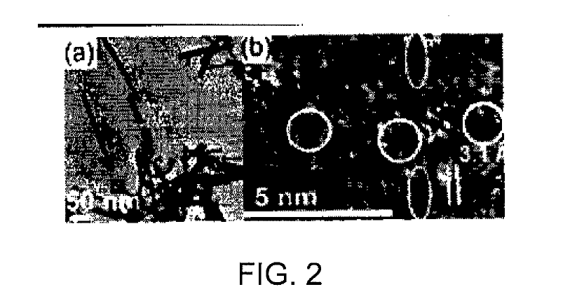

FIG. 2A shows an image of the detailed structures of CeO₂ nanorod obtained by transmission electron microscopy (TEM) with a Tecnai G2 F20 S-Twin microscope (available from FEI, Hillsboro, Oreg.) operated at 200 keV. The sample was prepared by drop-casting a solution of the sample sonicated, for no more than 5 seconds, in methanol onto a holey carbon film on a copper grid support.

FIG. 2B shows an enlarged image of a portion of the image of FIG. 2A. Locations of oxygen vacancy defects are represented by the circles and line segments.

Oxygen vacancy defects that can be identifiable by HRTEM include oxygen vacancy defect sites with missing oxygen atoms, and the linear cluster defect composed of lines of missing oxygen atoms in the first atomic surface layer. The images also show that rapid oxidation of Ce(OH)₃ results in a surface with many defects, and that oxygen vacancies were formed during annealing under vacuum.

The HRTEM indicated that the cerium oxide nanotubes have a wide variety of different types of defect sites. The defect sites that can be identified in the HRTEM images include step edge, grain boundary, and line defect sites. Also identifiable are the three major types of vacancy cluster defects: surface vacancy, subsurface vacancy, and linear vacancy clusters that represent both mobile and stable defects. These defects and vacancy clusters are likely significant contributors to the increased catalytic activity of the cerium oxide nanotubes and are correlated to the ratio of Ce³⁺ to Ce⁴⁺.

While it appears that the grain boundary and step edge defects appear during the synthetic processing, changing from simple hexagonal lattice to that of a fluorite structure, the appearance of the vacancy clusters seems to be largely due to the post processing. The activation of the cerium oxide nanotubes at low pressure (e.g., <0.1 Torr) and high temperature (e.g., 400° C.) appears to have introduced many vacancy cluster defects into the cerium oxide nanotubes. While HRTEM is only truly sensitive to columns of atoms, it is possible to detect areas which correspond to vacancy clusters.

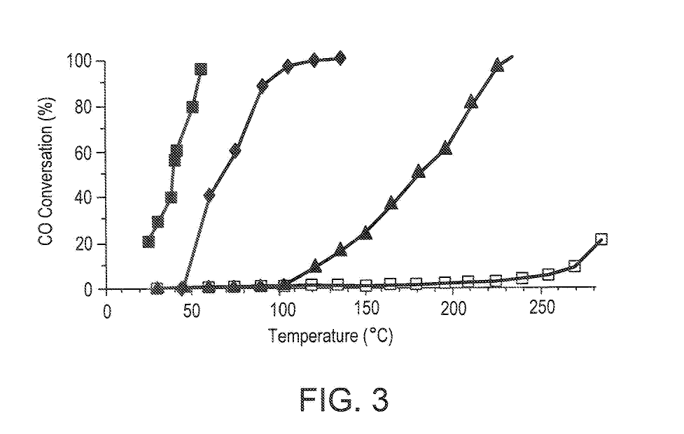

FIG. 3 is a graph showing the carbon monoxide conversion efficiency of metal cluster decorated cerium oxide at various temperatures. Data curves represent the carbon monoxide conversion efficiency of 0.01% Pd/CeO₂, 0.01% Au/CeO₂, CeO₂ nanotubes, and cerium oxide in bulk form, respectively. The curves indicate that metal cluster decorated cerium oxide nanotubes have a higher conversion efficiency than cerium oxide nanopowder, which has a higher conversion efficiency than cerium oxide in bulk form.

Long-term testing has demonstrated that the metal cluster cerium oxide nanotubes remain active over a period of at least 100 hours with almost no change in catalytic activity (data not shown). This is an additional indicator that the metal cluster cerium oxide nanotubes are acting as a catalyst rather than as a reactant.

When the cerium oxide is used as a catalyst for converting carbohydrate polymers to chemical products such as glucose, it may be preferable to use cerium oxide having a higher percentage of Ce³⁺ (e.g., 70%), while in other reactions it may be preferable to use cerium oxide having a lower percentage of Ce³⁺ (e.g., 40%). By adjusting a combination of processing conditions when fabricating the cerium oxide, the percentage of Ce³⁺ in the cerium oxide can be tuned to different values ranging from, e.g., 40% to 70%, that can be suitable for different applications. In one embodiment, a Ce³⁺ percentage greater than 45% is preferable. The conditions that can be adjusted include the pressure used in the low pressure activation, and the type of particles used to decorate the cerium oxide. If more than one type of particles isused to decorate the cerium oxide, the order in which the different types of particles are added to the cerium oxide during the decoration process may also affect the percentage of Ce³⁺ in the final cerium oxide product

The following describes examples for generating fluorite structured cerium oxide nanotubes having different levels of Ce³⁺.

For example, fluorite structured cerium oxide having approximately 40 atomic percent Ce³⁺ can be produced by activating the cerium oxide nanotubes at pressures at or below 0.1 Torr under flowing air or air like mixtures of gasses.

Fluorite structured cerium oxide having approximately 50 atomic percent Ce³⁺ can be produced by decorating the cerium oxide nanotubes with 2 atomic percent palladium, and activating the palladium decorated cerium oxide nanotubes at a pressure below 0.1 Torr in nitrogen.

Fluorite structured cerium oxide having approximately 60 atomic percent Ce³⁺ can be produced by decorating the cerium oxide nanotubes with 1 atomic percent tin, and activating the tin-decorated cerium oxide nanotubes at a pressure below 0.1 Torr in air. Fluorite structured cerium oxide having approximately 70 atomic percent Ce³⁺ can be produced by decorating the cerium oxide nanotubes with 0.01 atomic percent tin, and activating the tin decorated cerium oxide nanotubes at a pressure below 0.1 Torr in air.

Fluorite structured cerium oxide having greater than 70 atomic percent Ce³⁺ can be produced by decorating the cerium oxide nanotubes with 0.01 atomic percent tin, and activating the tin decorated cerium oxide nanotubes at a pressure below 0.05 Torr in nitrogen.

Each of the examples above can be further fine-tuned to adjust the percentage of Ce³⁺ (e.g., to achieve 45, 55, or 65 atomic percent Ce³⁺) designed to catalyze conversion of carbohydrate polymers to monosaccharides, as described herein.

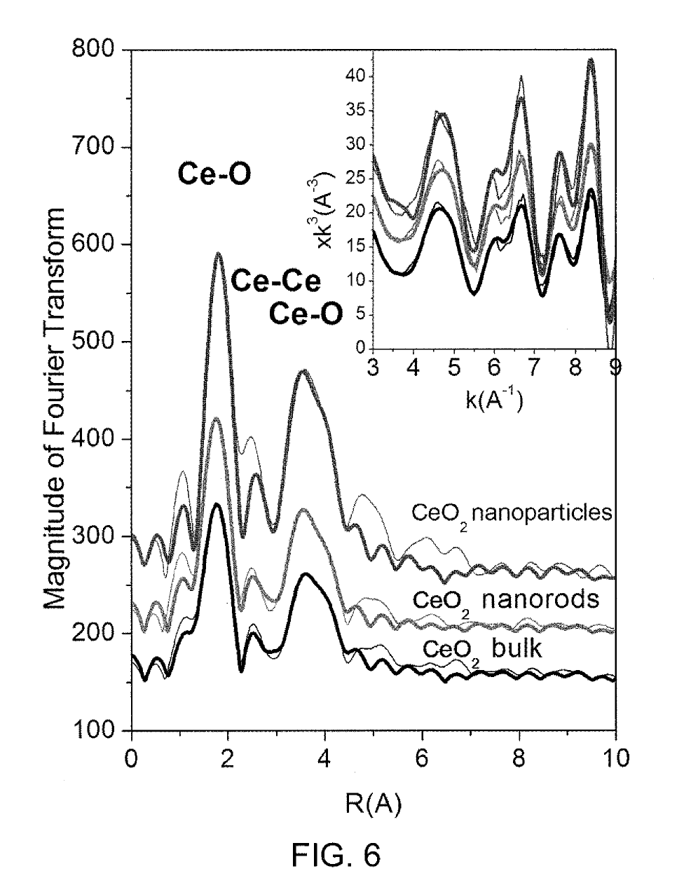

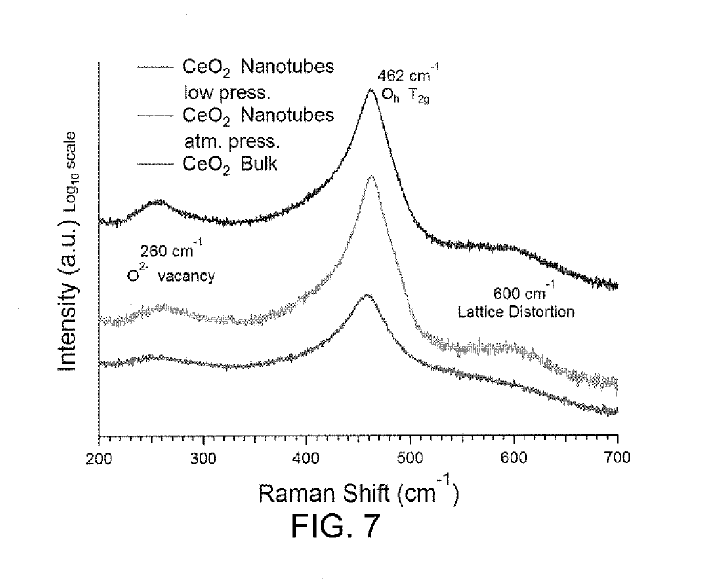

Although some examples have been discussed above, other embodiments and applications are also within the scope of the following claims. For example, measurement of the ratio of Ce³⁺ to Ce³⁻+Ce⁴⁺ can be indirectly inferred using scanning tunneling microscopy (STM) and high resolution transmission electron microscopy (HRTEM), which can both observe the density and types of oxygen vacancy defects that are positively correlated to the Ce^{3|} fraction. Extended x-ray absorption fine structure spectroscopy (EXAFS) can be used as it provides a direct measurement of many parameters of the crystal structure and is highly unlikely to change the oxidation states of cerium. For example, EXAFS data for bulk CeO₂ and CeO₂ nanoparticles and nanorods is shown in FIG. 6. Raman scattering spectroscopy can be used to indirectly measure the presence of oxygen vacancy defects which correlate positively to the Ce³⁺ fraction and can be used to quickly and inexpensively infer the presence of high density of Ce³⁺ in a cerium oxide sample. Raman scattering data of bulk CeO₂ and CeO₂ nanoparticles and nanorods is shown in FIG. 7.

The values for the Ce³⁺ fraction of the cerium oxide described above are stable at least below 100° C. under atmospheric pressure. When the surrounding temperature is above 450° C., the cerium oxide lattice may begin to anneal and the Ce³⁺ fraction may change significantly compared to the original Ce³⁻ fraction measured at room temperature, as long as there is a continuous supply of reactants.

The cerium oxide described above has a fluorite structure, which has a crystal lattice similar to that of CaF₂, also known as Fm 3̅m alternately written as Fm3-m. The cerium oxide may also have other structures. The fluorite structured cerium oxide can be produced as, e.g., fluorite structured cerium oxide nanocubes, nanoparticles, nanorods, nanowires, nanostars, or complex nanoshapes.

The following describes an example in which cellobiose was treated with fluorite structured tin cerium oxide nanotubes. Note that various parameter values, such as the quantities of the materials, temperatures of the processes, and time durations of the processes, are provided as examples only, other values can also be used.

A 25 mL sample of cellobiose solution (50 mM) was added to a round-bottomed flask. The solution was adjusted to a pH of 2.5 using H₂SO₄. A solution containing fluorite structured 0.01% tin cerium oxide nanotubes (10% molar concentration) was added to the reaction, and the mixture was refluxed in a silicon oil bath for 24 hours. Aliquots were collected throughout the reaction and the reaction progress was monitored by HPLC-MS chromatography.

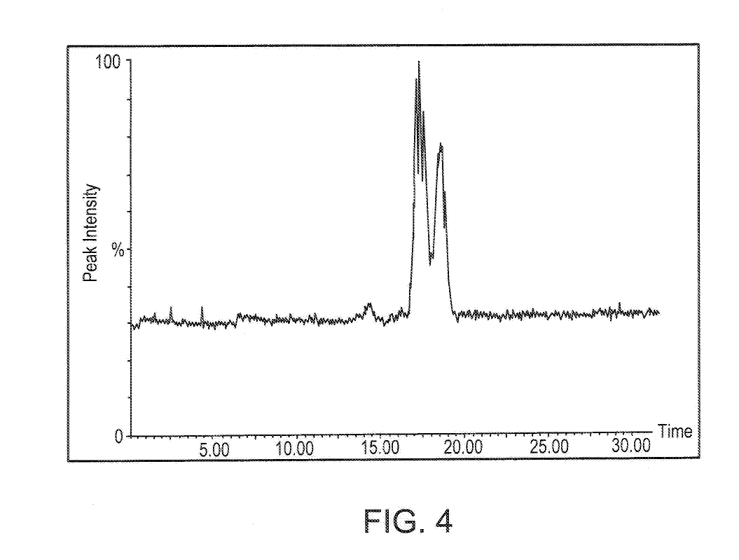

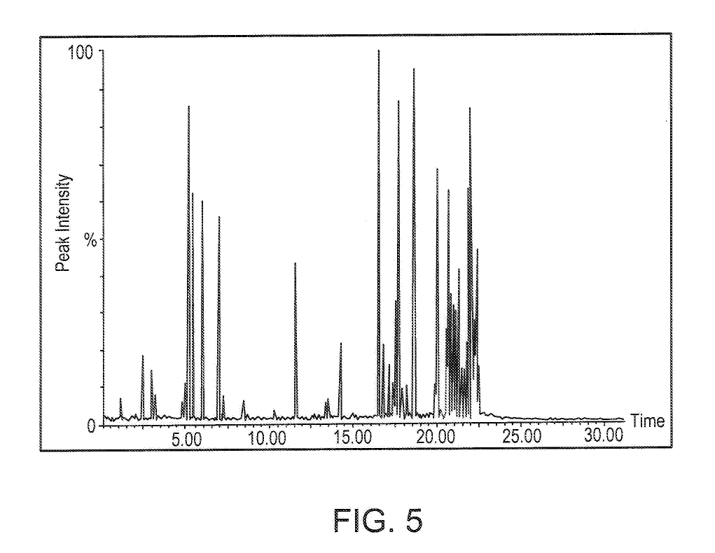

FIG. 4 is an HPLC-MS chromatograph of the cellobiose sample prior to addition of the catalyst. FIG. 5 is an HPLC-MS chromatograph of the cellobiose solution after 4 hours of digestion with 0.01% Sn/SO₄/CeO₂. As shown in FIG. 5, a range of reaction products are formed during the experiment, indicating that the catalyst is highly effective in degrading cellobiose.

Accordingly, in the drawings and specification, there has been disclosed a typical preferred embodiment of the invention, and although specific terms are employed, the terms are used in a descriptive sense only and not for purposes of limitation. The invention has been described in considerable detail with specific references to illustrated embodiments. It will be apparent, however, that various modifications and changes can be made within the spirit and scope of the invention as described in the forgoing specification and as defined in the appended claims.

What is claimed is:

1. A method for converting a carbohydrate polymer to a monosaccharide product, comprising the step of:
heating the carbohydrate polymer with a cerium oxide catalyst for a time sufficient to convert the carbohydrate polymer to the monosaccharide product,
the cerium oxide catalyst having a fluorite lattice structure comprising cerium atoms in mixed valence states of Ce³⁺ and Ce⁴⁺, in which the ratio of Ce³⁺/(Ce³⁺+Ce⁴⁺) in the lattice ranges from 40% to 90% at 20° C., the valence states Ce³⁺ and Ce⁴⁺ being reversible in reduction and oxidation reactions, the cerium oxide catalyst comprising small particles decorated near a surface of the fluorite structured cerium oxide lattice, in which the surface region of the cerium oxide lattice structure has a higher concentration of the small particles than an inner region of the cerium oxide lattice structure, the small particles having a diameter equal to or less than 1 nm.

2. The method of claim 1, wherein the carbohydrate polymer is cellulosic.

3. The method of claim 1, wherein the carbohydrate polymer comprises cellulose, hemicellulose, or cellobiose.

4. The method of claim 1, wherein the carbohydrate polymer comprises dextran, fructan or agarose.

5. The method of claim 1, wherein the monosaccharide product comprises glucose, fructose or galactose.

6. The method of claim 5, wherein the monosaccharide product comprises glucose.

7. The method of claim 6, wherein process further comprises a step of fermenting the glucose to form ethanol.

8. The method of claim 1, wherein the step of heating includes heating the carbohydrate polymer at a concentration of about 30-60 wt % in solution.

9. The method of claim 8, wherein the heating includes heating the carbohydrate polymer at a concentration of about 30 wt % in solution.

10. The method of claim 1, wherein the heating includes heating the carbohydrate polymer in an aqueous solution.

11. The method of claim 1, wherein the heating includes heating the carbohydrate polymer in a solution comprising an ionic liquid.

12. The method of claim 11, wherein the ionic liquid is selected from 1-ethlyl-3-methylimidazolium acetate ([EIMIM]acetate), 1-ethlyl-3-methylimidazolium bromide ([EIMIM]Br), 1-ethyl-3-methylimidazollum chloride ([EMINA]Cl), or 1-butyl-3-methylimidazolium chloride ([BMIM]Cl), and combinations thereof.

13. The method of claim 12, wherein the ionic liquid is 1-ethlyl-3-methylimidazolium acetate ([EIMIM] acetate).

14. The method of claim 1, wherein the heating includes heating the carbohydrate polymer at a temperature of about 100° C. to about 180° C. for a time period of about 0.01 hours to about 8 hours.

15. The method of claim 1, wherein the cerium oxide catalyst is a solid superacid.

16. The method of claim 1, wherein the cerium oxide catalyst is sulfated.

17. The method of claim 1, wherein the small particles comprise at least one of gold, silver, tin, palladium, platinum, an alloy of gold and silver, an alloy of gold and copper, the oxide of any of the above, or a combination of any of the above.

18. The method of claim 1, wherein the small particles comprise at least one of copper, zirconium, vanadium, iron, zinc, cobalt, silicon, nickel, manganese, rhodium, ruthenium, tungsten, rhenium, cadmium, iridium, molybdenum, phosphorus, tantalum, osmium, titanium, chromium, scandium, sulfur, rare earths elements, the oxide of at least one of the above, or a combination of the above.

19. The method of claim 1, wherein the small particles comprise tin particles, and the concentration of the tin particles on the cerium oxide ranges from 0.001 to 5.0 atomic percent compared to cerium.

20. The method of claim 19 wherein the concentration of the tin particles ranges from 0.005 to 0.02 atomic percent compared to cerium.

21. The method of claim 1, wherein the small particles comprise at least one of tin particles or tin oxide particles, and the concentration of the tin particles or tin oxide particles on the cerium oxide ranges from 0.1 to 5 atomic percent compared to cerium.

22. The method of claim 1 wherein the concentration of the small particles on the fluorite structured cerium oxide ranges from 0.001 to 5.0 atomic percent compared to cerium.

23. The method of claim 1 wherein the cerium oxide comprises cerium oxide nanoscale structures.

24. The method of claim 23 wherein the nanoscale structures comprise at least one of nanotubes, nanocubes, nanoparticles, nanorods, nanowires, nanostars or complex nanoshapes.

25. The method of claim 24, wherein the nanoscale structure comprises nanorods.

26. The method of claim 1, wherein the ratio of Ce³⁺/(Ce³⁻+Ce⁴⁻) in the lattice structure ranges from 40% to 50% at 20° C.

27. The method of claim 1, wherein the ratio of Ce³⁺/(Ce³⁻+Ce⁴⁻) in the lattice structure ranges from 50% to 60% at 20° C.

28. The method of claim 1, wherein the ratio of Ce³⁺/(Ce³⁻+Ce⁴⁻) in the lattice structure ranges from 60% to 70% at 20° C.

29. The method of claim 1, wherein the ratio of Ce³⁺/(Ce³⁻+Ce⁴⁻) in the lattice structure ranges from 70% to 90% at 20° C.

30. The method of claim 1, wherein the cerium oxide is capable of maintaining effective catalytic ability at temperatures at least up to 450° C.

31. A method of fabricating a catalyst, the method comprising:
fabricating fluorite structured cerium oxide having a lattice structure comprising cerium atoms in mixed valence states of Ce³⁺ and Ce⁴⁺;
decorating the cerium oxide with small metallic particles near a surface of the lattice structure such that a surface region of the cerium oxide lattice structure has a higher concentration of the small particles than an inner region of the cerium oxide lattice structure, the small particles having a diameter less than 1 nm;
sulfating the cerium oxide; and
activating the cerium oxide, in which after activation, the ratio of Ce³⁺/(Ce³⁺+Ce⁴⁻) in the cerium oxide lattice structure ranges from 40% to 90% at 20° C., the valence states Ce³⁺ and Ce⁴⁺ being switchable in reduction and oxidation reactions.

32. The method of claim 31, wherein decorating the cerium oxide with metallic small particles comprises decorating the cerium oxide with at least one of gold, silver, tin, palladium, platinum, an alloy of gold and silver, an alloy of gold and copper, the oxide of any of the above, or a combination of any of the above.

33. The method of claim 30, wherein decorating the cerium oxide with small particles comprises decorating the cerium oxide with at least one of copper, zirconium, vanadium, iron, zinc, cobalt, silicon, nickel, manganese, rhodium, ruthenium, tungsten, rhenium, cadmium, iridium, molybdenum, phosphorus, tantalum, osmium, titanium, chromium, scandium, sulfur, rare earths elements, the oxide of at least one of the above, or a combination of the above.

34. The method of claim 31, wherein the decorating the cerium oxide with small particles comprises decorating the cerium oxide with tin particles, the concentration of the tin particles on the cerium oxide ranging from 0.001 to 5.0 atomic percent compared to cerium.

35. The method of claim 34 wherein the concentration of the tin particles ranges from 0.005 to 0.02 atomic percent compared to cerium.

36. The method of claim 31, wherein the decorating the cerium oxide with small particles comprises decorating the cerium oxide with at least one of tin particles or tin oxide particles, and the concentration of the tin particles or tin oxide particles on the cerium oxide ranging from 0.1 to 5 atomic percent compared to cerium.

37. The method of claim 31, wherein the concentration of the small particles on the fluorite structured cerium oxide ranges from 0.001 to 5.0 atomic percent compared to cerium.

38. The method of claim 31, comprising mixing the cerium oxide with a solution containing a metal in an oxidized state to facilitate an auto-reduction reaction that produces metallic particles that decorate the surface of the cerium oxide.

39. The method of claim 31, comprising mixing the cerium oxide with a solution containing tin in an oxidized state to facilitate an auto-reduction reaction that produces metallic tin particles that decorate the surface of the cerium oxide.

40. The method of claim 39, wherein mixing the cerium oxide with a solution containing tin in an oxidized state comprises mixing the cerium oxide with a tin chloride solution.

41. The method of claim 31, wherein producing cerium oxide comprises producing cerium oxide nanoscale structures.

42. The method of claim 41 wherein producing cerium oxide nanoscale structures comprises producing at least one of nanotubes, nanocubes, nanoparticles, nanorods, nanowires, nanostars or complex nanoshapes.

43. The method of claim 42, wherein producing cerium oxide nanscale structures comprises producing cerium oxide nanorod structures.

44. The method of claim 31, wherein sulfating the cerium oxide comprises treating the cerium oxide with sulfuric acid.
